(19) United States
(12) Patent Application Publication
(10) Pub. No.: US 2003/0158522 A1
(43) Pub. Date: Aug. 21, 2003

(54) Device for administering an injectable product

(76) Inventor: Serge Huegli, Kirchberg (CH)

Correspondence Address: David E. Bruhn, DORSEY & WHITNEY LLP, Intellectual Property Department, 50 South Sixth Street, Suite 1500, Minneapolis, MN, 55402-1498, US

(21) Appl. No.: 10/368,989

(22) Filed: Feb. 19, 2003

(30) Foreign Application Priority Data
Feb. 19, 2002 (DE) 102 06 910.7

(51) Int. Cl. A61M 37/00

(52) U.S. Cl. 604/133

(57) ABSTRACT

A device for administering an injectable product including an accommodating device for an ampoule and a drive mechanism operably coupled to the ampoule in order to administer the contents of the ampoule, wherein the drive mechanism is provided in the accommodating device in such a way that in an initial state, when the ampoule is inserted, at least one part of the drive mechanism is arranged in the interior of the ampoule.

### CROSS-REFERENCE TO RELATED APPLICATIONS

This application claims priority to German Application No. DE 102 06 910.7, filed on Feb. 19, 2002, which is incorporated herein by reference.

### BACKGROUND

The invention relates to a devices for administering injectable products. More particularly, it relates to devices for administering an injectable product in doses, for example, infusion pumps, wherein an injectable product contained in an ampoule inserted into the device can be dispensed in doses.

FIG. 4 shows, by way of example, a known ampoule 5 arranged in an infusion pump 1, the ampoule being completely filled. The stopper 4 seals off the filling volume of the ampoule 5 on the side of the ampoule 5 opposite the output opening 5a. The drive mechanism is arranged adjacent to the stopper 4 and is formed by a non-rotationally mounted piston rod 3 comprising an inner thread and a threaded rod 2 which engages with the inner thread of the piston rod 3 and can for example move the piston rod 3 towards the output opening 5a of the ampoule 5 when it is rotated, thus moving the stopper 4 into the ampoule 5 and displacing from the ampoule 5 a substance present in the ampoule 5, in order to be able to dispense the substance in doses. As may be seen from FIG. 4, the overall height or length of the arrangement shown is equal to the length of the ampoule 5 plus the length of the drive mechanism 17.

In general, various infusion pumps are known into which a filled ampoule having standardized dimensions can be inserted in order to dispense a substance contained in the ampoule in doses with the aid of a drive mechanism. The dimensions of the standardized ampoule, together with the dimensions of the drive mechanism, dictate a certain minimum height of infusion pumps, which accordingly cannot be further miniaturized than the minimum of the length of the ampoule plus the height of the drive mechanism. Conventionally, standardized 3.15 ml ampoules are used.

A smaller ampoule could in principle be constructed. However, the time frame for developing and officially admitting or permitting such an ampoule amounts to several years. Moreover, ampoules should exhibit the greatest possible compatibility, i.e., should not specifically be designed only for an individual type of device or a particular application, in order to save the cost, time and staffing requirements for developing, registering and marketing a new type of ampoule, and in order to be able to retain systems for the known standardized ampoule which are available or already in planning.

### SUMMARY

It is an object of the present invention to provide a device for administering an injectable product which is smaller than known devices, but which uses standard ampoules.

In accordance with a first embodiment, the device for administering an injectable product in accordance with the invention, for example an infusion pump or an injection device, comprises an accommodating device for the ampoule, wherein the ampoule is guided into the accommodating device and held in a defined position by the administering device, in order to dispense a substance contained in the ampoule, preferably in doses. Furthermore, a drive mechanism is provided which may be coupled to the ampoule in order to dispense the contents of the ampoule. A shifting stopper is provided in the ampoule, which can be connected or operably coupled to the drive mechanism, in order to push the stopper into the ampoule via the drive mechanism and so dispense a substance contained in the ampoule. In accordance with the invention, the drive mechanism is provided in the accommodating device of the administering device in such a way that in the initial state, i.e., before a first administering process from a newly inserted ampoule, at least one part of the drive mechanism protrudes into the interior of the ampoule. Consequently, the ampoule to be inserted into the administering device must not be completely filled, so as to create an empty space in the ampoule into which the drive mechanism of the administering device can be inserted. Accordingly, it is possible to reduce the overall height of the administering device below the minimum height or size achievable up until now, which has been defined by the length of the ampoule plus the height of the drive mechanism in the initial state, i.e., for example, when the piston rod is retracted. If the drive mechanism is inserted into an incompletely or partially filled ampoule, then the height or length of the drive mechanism can also be reduced, since the drive mechanism no longer has to pass along the entire length of the ampoule, but only the length of the ampoule minus the height of the drive mechanism, in order to be able to completely dispense the substance contained in the ampoule.

In some embodiments, 3.15 ml ampoules are preferably used, having an overall length of about 57.6 mm and an inner diameter of approximately 12 mm and a length of the inner space of the ampoule of about 40 mm, the inner space of the ampoule being either filled with a substance or at least partially used for accommodating a dispensing mechanism.

In some embodiments, particularly advantageously the ampoule is only filled about half full, such that the drive mechanism can be roughly as high or as long as half the ampoule length.

In general, the height of the drive mechanism in the initial state can be a selected fraction of the ampoule length, such as, for example, about a third, a quarter or even an eighth of the ampoule length. Then, by using a telescopic or other suitable mechanism, the drive mechanism passes along the entire length of the ampoule in order to be able to completely dispense the contents of the ampoule.

In accordance with another aspect of the invention, an administering device is proposed which comprises an accommodating device for an ampoule and a drive mechanism which may be coupled to the ampoule to dispense the contents of the ampoule. The drive mechanism can then maximally pass along a partial region of the ampoule, making it possible to keep the height or length of the drive mechanism relatively small. In order to be able to completely dispense the contents of the ampoule, at least one adapter or attachment is provided on the administering device in accordance with the invention, wherein in some embodiments, said adapter or attachment can preferably be removed from the administering device such that, when the at least one adapter is present, the ampoule is inserted into the administering device in such a way that the drive mechanism can pass along a rear partial region of the ampoule to dispense a first part of the contents of the ampoule, and when the at least one adapter is removed, the ampoule can be inserted further into the administering device, such that the drive mechanism, which is preferably returned to the initial state, protrudes at least to a certain extent into the interior of the ampoule. This enables a second or further part of the contents of the ampoule to be successively dispensed using the drive mechanism.

If, for example, an adapter or attachment is used which enables the ampoule to be inserted into the administering device in such a way that the drive mechanism can pass along the lower or rear half of the ampoule length, then when the adapter is removed the ampoule can be inserted into the administering device in such a way that the drive mechanism lies in roughly the lower half of the ampoule. This enables the second part of the contents of the ampoule to be dispensed and the ampoule to thus be substantially completely emptied.

In some embodiments, the adapter or attachment can also advantageously be formed in or comprise a number of parts or stages, such that it is possible to insert the ampoule into the administering device in stages, enabling the contents of the ampoule to be dispensed in a number of steps.

In some embodiments, the attachment and the administering device are large enough that the ampoule inserted partly into the adapter and partly into the administering device lies roughly adjacent to the drive device in the administering device and protrudes by about the length of the administering device, such that the ampoule can be inserted completely into the administering device to dispense the last part of the contents.

In accordance with a further aspect of the invention, a device for administering an injectable product is provided, comprising an accommodating device for an ampoule and an ampoule slaving means or an ampoule transport device in order to move the ampoule relative to the administering device, in some embodiments to be able to retract or draw the ampoule into the administering device. The ampoule can, for example, then be drawn or pushed into the administering device by hand, to which end a pushing mechanism or a rotatable element can be provided on the administering device, using which the ampoule can be moved into or out of the administering device when the ampoule is to be shifted or exchanged. It is also possible to draw the ampoule into the administering device or push it out automatically, for example by means of an electric motor or other suitable means.

The movement of the ampoule relative to the administering device enables the contents of the ampoule to be dispensed completely using a drive mechanism which cannot pass along the complete ampoule length, since once the drive mechanism has been maximally extended and a first part of the contents of the ampoule has been dispensed, the drive mechanism can be returned, i.e., returned to the initial state and at the same time or subsequently, the ampoule can be moved towards the drive mechanism in order to dispense a second part of the contents of the ampoule.

In the variants described above and other embodiments of the administering device in accordance with the invention, a piston rod can be provided as the drive mechanism, wherein the piston rod can be attached to a stopper which is movable in the ampoule or coupled to the stopper in order to gradually push the stopper into the ampoule and displace a substance from the interior of the ampoule and thus dispense said substance outwards. The piston rod is advantageously coupled to a threaded rod via an inner thread, the threaded rod engaging with the piston rod and, when it is rotated, advancing or retracting the piston rod, which is advantageously mounted non-rotationally in the administering device. To this end, a motor can be provided, for example, an electric motor, which rotates the thread spindle and thus moves the piston rod into the interior of the ampoule, thereby pushing the stopper into the ampoule, in order to displace and dispense the substance contained in the ampoule. Such drive mechanisms and others that may be suitable are known in the prior art.

By using the teaching in accordance with the invention, it is possible to design smaller drive mechanisms, e.g., to reduce the length of the piston rod and the thread spindle, enabling the overall height or size of the administering device, for example an infusion pump, to be reduced.

In accordance with another aspect, the present invention relates to an ampoule, preferably a standardized 3.15 ml ampoule, which is not completely filled. In particular, the invention relates to an ampoule which is only about half, e.g., with 1.5 ml, or two-thirds full, creating an empty space in the ampoule in which the drive mechanism of the administering device can be arranged in order to reduce the overall height of the administering device.

In general, ampoules which can be re-filled may be used, e.g., by returning the drive or dispensing mechanism when inserted in the administering device.

### BRIEF DESCRIPTION OF THE DRAWINGS

The invention will now be described by way of various exemplary embodiments.

FIG. 1 depicts a first embodiment of an infusion pump drive mechanism in accordance with the invention;

FIG. 2 depicts a second embodiment of an infusion pump in accordance with the invention, comprising an adapter;

FIGS. 3A and 3B depict a third embodiment of an infusion pump in accordance with the invention, comprising an ampoule which may be drawn in automatically; and

FIG. 4 depicts a drive mechanism of a known infusion pump.

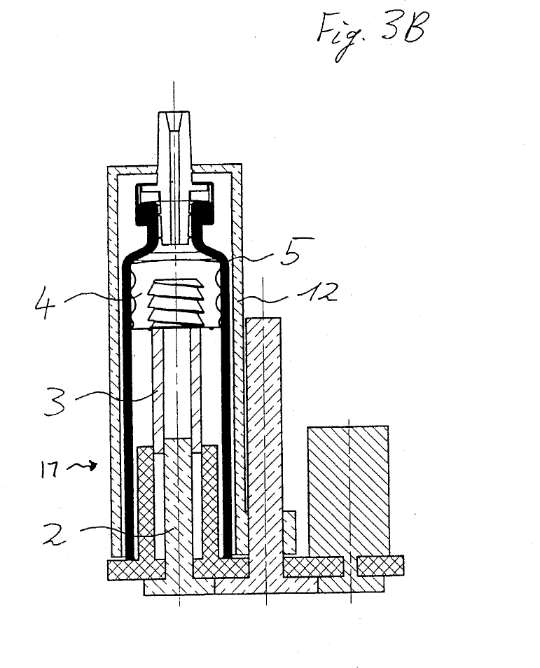

### DETAILED DESCRIPTION

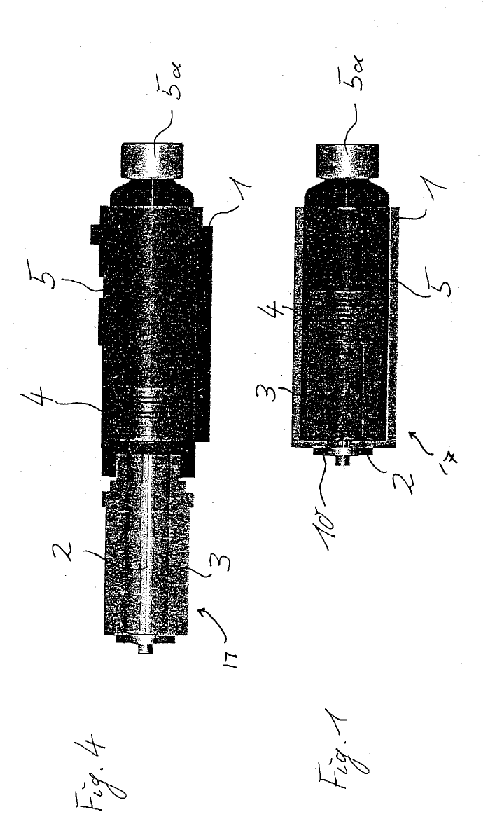

FIG. 1 schematically shows a drive mechanism 17, comprising a threaded rod 2 and a piston rod 3, of an infusion pump 1 for dispensing a substance contained in an ampoule 5. The ampoule 5 is about half full and at its front end comprises a dispensing opening 5a through which the substance contained in the ampoule 5, such as for example insulin, hormone or other substances, are dispensed to a user in doses, for example via a catheter and a port body. The filled region of the ampoule 5 is sealed off by a stopper 4 which can be moved through the ampoule in the longitudinal direction and which, when moved to the right in the arrangement shown in FIG. 1, displaces the substance contained in the ampoule, such that said substance is dispensed through the dispensing opening 5a. The piston rod 3 lies adjacent to the stopper 4, said piston rod being for example guided non-rotationally in the longitudinal direction of the ampoule 5 by lateral guides. The piston rod 3 has an inner thread with which the outer thread of a threaded rod 2 engages, wherein said threaded rod can move the piston rod 3 back and forth by being rotated, in order for example to dispense a substance from the ampoule 5 or to return the piston rod 3 to the initial position shown. The threaded rod 2 and the piston rod 3 are advantageously about the same length, such that in the initial state, i.e., when the piston rod 3 is completely retracted, the threaded rod 2 is completely accommodated by the piston rod 3, which defines the height of the drive mechanism. When the piston rod 3 is completely extended, the overall length of the drive mechanism 17 formed by the threaded rod 2 and the piston rod 3 is roughly the length of the threaded rod 2 plus the length of the piston rod 3, such that, in the embodiment shown in FIG. 1, a drive mechanism 17 reaching about halfway into the ampoule 5 is sufficient in order to be able to completely empty the half-filled ampoule 5. The threaded rod 2 can be coupled to an electric motor (not shown) which can be arranged inside the infusion pump 1 next to the threaded rod 2, so as not to increase the height further. The threaded rod 2 can for example be coupled to an electric motor via a gear 10 fixed to the threaded rod 2.

An infusion pump comprising the drive mechanism 17 shown in FIG. 1 can thus exhibit a height or length which is as large or preferably not substantially larger than the length of the ampoule 5, such that a miniaturized infusion pump 1 can be achieved in accordance with the invention.

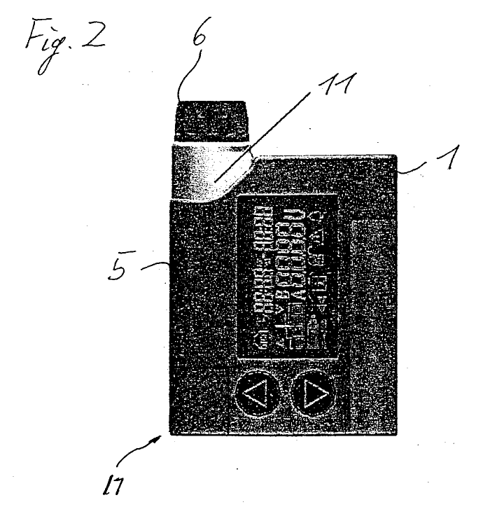

FIG. 2 shows another embodiment of the invention, in which an attachment 11 is provided on the infusion pump 1 which enables an ampoule to plugged, only partially, into the infusion pump 1. In the initial position, the drive mechanism 17 is still arranged outside the ampoule 5 and can dispense a first part of the contents of the ampoule 5. If the first part of the contents of the ampoule 5 has been dispensed, then the attachment 11 can be removed and the ampoule 5 can be completely inserted into the infusion pump, such that the drive mechanism 17 is arranged inside the ampoule 5. A second part of the contents of the ampoule can then be dispensed. By using an adapter or attachment 11, a completely filled ampoule 5 can also be completely emptied using a drive mechanism 17 exhibiting a reduced height. Due to the reduced height of the drive mechanism 17 which for example can only pass along half the ampoule length, the overall height of the infusion pump 1 can be reduced. Using a securing screw 6, the ampoule 5 inserted into the infusion pump 1 can be secured against falling out.

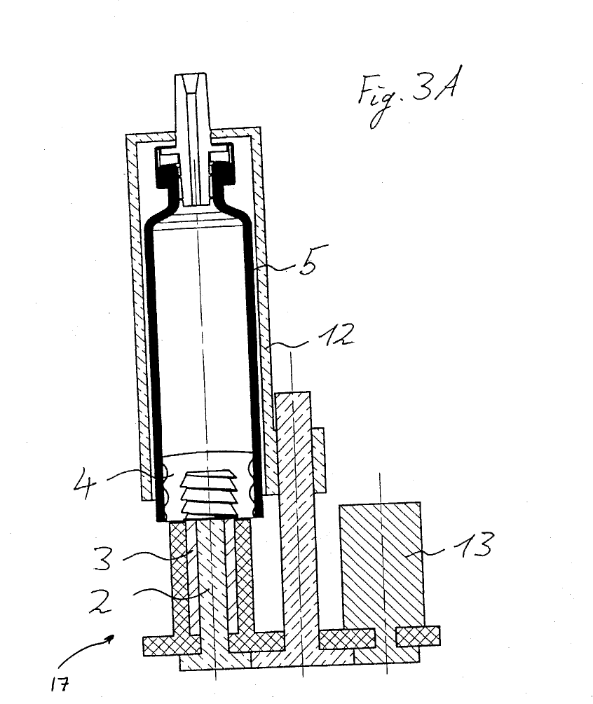

FIG. 3 shows a third embodiment of an infusion pump 1 in accordance with the invention, comprising an ampoule 5 which may be retracted and extended automatically. A completely filled ampoule 5 can then be coupled or connected to an ampoule slaving means 12, so as to be able to draw the ampoule 5 into the infusion pump 1 or push the ampoule 5 out of the infusion pump 1 manually or automatically, for example by means of an electric motor 13. As shown in FIG. 3A, an ampoule 5 similar to the configuration shown in FIG. 2 is incompletely inserted into the infusion pump 1. A first part of the contents of the ampoule 5 can be dispensed by the drive mechanism 17. Once the first part of the contents of the ampoule 5 has been dispensed, the drive mechanism 17 can be moved back to the initial state, wherein at the same time or subsequently in time, the ampoule 5 can be drawn further into the infusion pump 1 by the ampoule slaving means 12. FIG. 3B shows the ampoule 5 drawn in completely. Another part of the of the contents of the ampoule can then be dispensed.

It is also possible to dispense the contents of the ampoule alone by shifting the ampoule 5 by means of the ampoule slaving means 12, without having to move the drive mechanism 17 for this purpose. As an alternative to using a drive mechanism 17, it is even possible to simply provide a displacing element in the infusion pump 1 which, when the ampoule 5 is pushed in, pushes the stopper 4 relative to the ampoule 5, towards the front end of the ampoule 5, so as to displace a substance contained in the ampoule 5 and thus dispense said substance from the ampoule.

Alternatively, a combination of operating the ampoule slaving means 12 and the drive mechanism 17 can also be used to dispense a particular amount of a substance from the ampoule

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering an injectable product, comprising an accommodating device for an ampoule and a drive mechanism operably coupled to said ampoule in order to administer the contents of the ampoule, wherein said drive mechanism is provided in said accommodating device in such a way that in the initial state, when the ampoule is inserted, at least a portion of the drive mechanism is in the ampoule.

2. The administering device as set forth in claim 1, wherein at least an eighth of the drive mechanism is arranged in the interior of the ampoule in the initial state.

3. The administering device as set forth in claim 2, wherein more than an eighth of the drive mechanism is arranged in the interior of the ampoule in the initial state.

4. A device for administering an injectable product, comprising an accommodating device for an ampoule and at least one attachment enabling the ampoule to be inserted into said administering device by only a fraction of the ampoule length.

5. The administering device as set forth in claim 4, wherein said attachment may be removed from the administering device.

6. The administering device as set forth in claim 4, wherein the attachment comprises more than one attachment part.

7. The administering device as set forth in claim 6, wherein said attachment parts can be removed individually.

8. The administering device as set forth in claim 4, wherein the attachment is large enough that the ampoule protrudes from the administering device by the length of a drive mechanism.

9. A device for administering an injectable product, comprising an accommodating device for an ampoule comprising an ampoule slaving means for moving the ampoule relative to the administering device.

10. The administering device as set forth in claim 9, wherein said ampoule slaving means can be operated manually.

11. The administering device as set forth in claim 9, wherein said ampoule slaving means can be operated automatically.

12. The device as set forth in claim 1, wherein the drive mechanism comprises a piston rod and a threaded rod which engages said piston rod.

13. The device as set forth in claim 1, wherein the drive mechanism comprises a telescopic mechanism comprising at least two generally concentric shifting elements.

14. The device as set forth in claim 13, wherein the drive mechanism comprises a telescopic mechanism comprising three generally concentric shifting elements.

15. An ampoule which comprises an empty space in its interior to accommodate a drive mechanism.

16. A device for administering an injectable product comprising an accommodating device for an ampoule containing the injectable product and a drive mechanism operably coupled to the ampoule, wherein the drive mechanism is associated with the accommodating device in such a way that when the ampoule is initially accommodated by the accommodating device at least a portion of the drive mechanism is in the ampoule.
